(19) United States
(12) Patent Application Publication
(10) Pub. No.: US 2023/0055314 A1
(43) Pub. Date: Feb. 23, 2023

(54) Blood Collection Apparatus

(71) Applicant: Elizabeth Deckler, Miami, FL (US)

(72) Inventor: Elizabeth Deckler, Miami, FL (US)

(21) Appl. No.: 17/407,315

(22) Filed: Aug. 20, 2021

(51) Int. Cl. A61B 5/15; B01L 3/00

(52) U.S. Cl. CPC A61B 5/150343; A61B 5/150145; B01L 3/5082; B01L 2300/04; B01L 2300/1883; B01L 2300/06

(57) ABSTRACT

A blood collection apparatus comprising: (i) a test tube for storing blood extracted from a patient, the test tube comprising a vacuum facilitating an extraction of blood; (ii) a heat transfer element encapsulating the test tube and storing at least two reagents capable of initiating a heat transfer process contemporaneously with the extraction of the blood, the heat transfer element further comprising a fracturable element that when fractured enables the at least two reagents to initiate the heat transfer process; and (iii) an insulation element encapsulating the heat transfer element, the insulation element inhibiting a loss of a temperature change of the blood, wherein test tube is removable, contemporaneously with an initiation of a blood test, from the insulation element encapsulating the heat transfer element, and wherein the apparatus comprises a circumference suitable to be mated with a general purpose test tube needle holder.

### BACKGROUND OF THE INVENTIONS

### 1. Field Of The Inventions

This disclosure relates to a blood collection apparatus. More specifically, this disclosure relates to apparatuses for and methods of collecting blood into a test tube at a pre-determined temperature.

### 2. Description Of The Related Art

Current methods of drawing blood from a patient utilize a blood collection apparatus that does not comprise a heat transfer process to either cool or warm the blood as it is drawn into a test tube. Prior art test tubes and vacuum tubes such as manufactured by Becton, Dickinson and Company do not incorporate the capability for an endothermic or exothermic reaction to cool or warm the blood as it enters the test tube.

For example, U.S. Patent Application Publication No. 20100254859A1 of Chiarin published on Feb. 21, 2012 discloses to a test tube made of plastic designed for taking blood samples. U.S. Patent Application Publication No. US20070125677A1 of Oronsky published on Jun. 7, 2007 discloses a thermal and/or light protective container assembly. U.S. Pat. No. 6,971,506B2 issued to Hassinen on Dec. 6, 2005 discloses a test tube carrier that assists with transport of a test tube. U.S. Pat. No. 6,467,299 issued to Coetzee on Oct. 22, 2002 is a container for a vial or ampoule designed to maintain a desired temperature. These and similar references do not integrate the capability for an endothermic reaction or exothermic reaction to cool or warm the blood as it enters the test tube.

### BRIEF SUMMARY OF THE INVENTIONS

In the United States alone, 13 billion blood tests are done every year. Blood ammonia level, a common blood test drawn to evaluate patients with liver disease, must be placed in an ice slurry once drawn. Blood and plasma analysis of catecholamines, metanephrines, pyruvate, lactic acid, angiotensin converting enzyme, ACTH, acetone, free fatty acids, renin activity, and vasoactive peptide all require the sample be chilled, typically in an ice slurry, after being drawn. Blood drawn for cryoglobulin analysis requires the sample be placed on a heating block. Delayed or omitted temperature control of blood samples can alter the results of analyses. These alterations can be at best costly and at worst deadly. In particular, ammonia increases in the blood every second it is kept at room temperature, and a falsely elevated blood ammonia level could lead to an unnecessary medical workup, additional days in the hospital, or a deadly oversight for a hospitalized patient.

The current method of drawing blood samples requires immersing the test tube with the blood sample in a bath of ice is inexcusably time intensive and prone to error. The current practice involves a nurse, phlebotomist, or other healthcare worker to painstakingly gather a container of ice, draw the patient's blood, and then place the test tube in the ice bath. Nurses typically take care of several patients at a time, and in an ICU setting where the blood ammonia level is often assessed, the time they spend gathering an ice bath could be better spent tending to the needs of other critically ill patients. Additionally, placing the samples in an ice bath may not always lead to correct results. Studies have shown that large pieces of ice do not cool the entire surface area of the test tube accurately. The time between drawing the blood into a room-temperature test tube and placing it in an ice bath can result in incorrect analyses by the laboratory.

The present embodiments of the invention advantageously solve the shortcomings inherent in the current method of obtaining and analyzing lab tests that require the sample to be cooled or heated. An embodiment of the disclosed blood collection apparatuses comprises a test tube for storing blood extracted from a patient, the test tube comprising a vacuum facilitating an extraction of blood from the patient, and further comprising a test tube septum. The blood collection apparatuses also comprise a heat transfer element encapsulating the test tube and storing at least two reagents capable of initiating a heat transfer process contemporaneously with the extraction of the blood from the patient, the heat transfer element further comprising a fracturable element that when fractured enables the at least two reagents to initiate the heat transfer process. In certain embodiments, the blood collection apparatuses further comprise an insulation element encapsulating the heat transfer element, the insulation element inhibiting a loss of a temperature change of the blood.

The disclosed blood collection apparatuses are designed to raise the standard of care. Advantageously, with the use of the disclosed blood collection apparatuses, lab tests will be more accurate and less time intensive to collect. The blood will be immediately drawn into a tube at the desired temperature. Eliminating the time spent gathering cooling or heating materials prior to drawing blood could save the health system an insurmountable amount of time and money. More accurate lab tests will result in fewer unnecessary tests, hospital days, and procedures. The blood collection apparatus will also decrease the risk of misdiagnosis.

### BRIEF DESCRIPTION OF THE DRAWINGS

FIG. 1 is a front section view and a cross section view of a blood collection apparatus shown in combination with a general purpose test tube needle holder, the apparatus comprising a test tube element and a heat transfer element, wherein a perimeter of the heat transfer element includes insulation, wherein the heat transfer element further comprises a fracturable element that is fractured with the application of a fracture force to enable at least two reagents to initiate an endothermic reaction or exothermic reaction.

FIG. 2 is a front section view and a cross section view of a blood collection apparatus comprising a test tube element and a heat transfer element, wherein the perimeter of the heat transfer element includes insulation, wherein the heat transfer element further comprises a fracturable element that is fractured with the application of a fracture force to enable at least two reagents to initiate an endothermic reaction or exothermic reaction, and wherein a fracture force is applied on the side of the blood collection apparatus.

FIG. 3 is a front section view and a cross section view of a blood collection apparatus comprising a test tube element and a heat transfer element, wherein the perimeter of the heat transfer element includes insulation, wherein the test tube element is a general purpose test tube shown in combination with a general purpose test tube needle holder.

FIG. 4 is a front section view and a cross section view of a blood collection apparatus comprising a test tube element and a heat transfer element, wherein the perimeter of the heat transfer element includes insulation, wherein the test tube element is a general purpose test tube, and wherein a fracture force is applied by the insertion of the test tube into the blood collection apparatus.

FIG. 5 is a front section view and a cross section view of a blood collection apparatus comprising a test tube element, heat transfer element, and an insulation element, shown in combination with a general purpose syringe.

FIG. 6 is a front section view and a cross section view of a blood collection apparatus comprising a test tube element and a heat transfer element, wherein the perimeter of the heat transfer element includes insulation, wherein a fracture movement is applied bidirectionally to cause a fracturing element to fracture the fracturable element.

FIG. 7 is a front section view and a cross section view of a blood collection apparatus comprising a test tube element, heat transfer element, and an insulation element, wherein a mixing movement causes a mixing element to mix the reagents in the heat transfer element.

FIG. 8 is a front section view and a cross section view of a blood collection apparatus comprising a test tube element, heat transfer element, and an insulation element, wherein the heat transfer element comprises a breakable ampoule containing at least one of the reagents.

FIG. 9 is a front section view and a cross section view of a blood collection apparatus comprising a test tube element, and a heat transfer element, wherein the heat transfer element comprises a heat transfer agent.

### DETAILED DESCRIPTION OF THE INVENTIONS

For purposes of the present disclosure, various terms used in the art are defined as follows:

The term “exemplary” shall mean “serving as an example, instance, or illustration.” Any aspect or embodiment described herein as “exemplary” is not necessarily to be construed as preferred or advantageous over other aspects or embodiments described herein.

The term “herein” shall mean in the entirety of this specification including drawings, abstract, and claims. The term herein is not limited to the paragraph, section, or embodiment in which it may appear.

The terms “include”, “comprise”, and “contains” do not limit the elements to those recited. By contrast, only the term “consist” limits the elements to those listed. Unless specifically stated otherwise, the term “some” refers to one or more.

The term “responsive” does not limit the elements, conditions, and/or requirements that may be taken into consideration. For example, an element or structure that is responsive to a specified requirement is not limited to being responsive to only that specified requirement. An element or structure may be responsive to a specified requirement and a second non-specified requirement, specially, when the second requirement, while described as an alternative requirement, may be also deemed complementary.

No conceptual distinction should be drawn from the use of the terms on, at, or in.

The term “adjacent” shall mean next to, encasing, housing, interacting with, or in close proximity of.

The terms “apparatus”, “device”, “instrument”, and “assembly” may be used herein interchangeably and are not intended to limit the scope of the disclosure.

The term “ampoule” shall mean a small vessel, container, capsule, and the like in which a substance, such as a reagent, is sealed, and capable of being fractured, broken, or cracked to release the substance.

The term “blood” shall mean blood, plasma, bodily fluid, and any substance extractable from a patient.

The term “blood test” shall mean test or procedure, such as a complete blood count, ammonia level, or comprehensive metabolic panel that is undertaken substantially after the extraction of blood from a patient into a test tube.

The term “button” shall mean an apparatus, which can be pressed, depressed, pushed, clicked, or activated that controls a mechanism or process.

The term “chemical reaction” shall mean a reaction or process that leads to the chemical transformation of one set of chemical substances to another and may be used interchangeably with the term “chemical process”. The terms “reaction” and “process” are interchangeable.

The term “element” shall mean an element, component, piece, part, section, and module. The terms “element”, “component”, “piece”, “part”, “section”, and “module” may be used herein interchangeably and are not intended to limit the scope of the disclosure.

The term “encapsulating” shall mean substantially, but not necessarily entirely, encapsulating, enclosing, surrounding, and covering.

The term “general purpose” shall mean not specially adapted in function or design to be used in combination with a blood collection apparatus.

The term “heat transfer element” shall mean an element, capsule, chamber, compartment, and partitioned space. The term “heat transfer” is synonymous with the term “heat exchange” and in certain embodiments a heat transfer absorbs heat and in alternate embodiments a heat transfer releases heat.

The term “insulation” shall mean a material, substance, coating, and mass used to inhibit a heat transfer. The inhibiting not necessarily requiring a totality in the elimination of heat transfer. The degree of inhibiting of the insulation being responsive to the quantity utilized and the insulating property of the insulation.

The term “needle” shall mean a hollow needle used to inject substances into a patient or extract blood from the patient. A needle may be an element of a butterfly needle, a component part of a test tube needle holder, and a pointed hollow end of a hypodermic syringe or instrument used to extract blood.

The term “patient” shall mean a human, animal, and object from which blood may be extracted.

The term “reagent” shall mean a substance or compound added to cause a chemical process or, in the case of a reactant, to be consumed in the course of a chemical process. A reagent herein comprises, for example, ammonium nitrate, barium hydroxide, urea, water, sodium acetate, iron, calcium chloride, magnesium sulfate, and ammonium chloride.

The term “substance” shall mean any matter and may be used interchangeably with the terms “chemical”, “water”, “liquid”, and “matter”.

The term “syringe” shall mean an instrument capable of drawing or extracting blood from a patient and ejecting blood into a blood chamber or test tube.

The terms “test tube element” and “test tube” are interchangeable and shall mean any test tube, culture tube, sample tube, blood collection tube, vacuum tube, instrument, device capable of storing blood extracted from a patient, general purpose test tube, and specially adapted test tube. The term “vacutainer” is a registered trademark of Becton, Dickinson & Company for a vacuum tube.

The term “test tube needle holder” shall mean a blood collection component used in conjunction with a vacuum tube. Examples of “test tube needle holders” include vacutainer holders and vacutainer hubs.

The term “test tube septum” shall mean any material of any composition that covers or seals the opening of a test tube and which may be puncturable. The term “test tube septum” shall also mean a test tube rubber stopper, test tube snap-top, and test tube hinge cap.

The term “user” and “practitioner” are used interchangeably and shall mean a nurse, phlebotomist, respiratory therapist, doctor, physician assistant, machines, veterinarian, veterinary assistant, and any other entity capable of extracting blood from a patient.

The term “vacuum” shall mean an enclosed space or chamber entirely devoid of matter, or from which matter, especially air, has been partially or entirely removed so that the matter or gas that may remain in the space exerts less pressure.

The term “vacuum tube” shall mean a blood collection test tube comprising a vacuum. A vacuum tube is usually a glass or plastic test tube with a rubber stopper sealing a vacuum inside of the tube and facilitating the drawing of a volume of blood.

The above defined terms and other terms explicitly defined herein are to be understood as defined in this document. Incorporation by reference shall not act to modify, limit, or broaden the definitions hereinabove provided or formally defined in this document. A term that is not formally defined in this document is defined herein to have its ordinary and customary meanings.

In the various embodiments disclosed herein, a blood collection apparatus comprising a test tube element and test tube septum. The test tube element is vacuum sealed to assist with drawing blood into the tube. Disclosures of a test tube and the use of vacuum sealing, which are incorporated herein by reference in their entirety, include U.S. Pat. No. 1,513,360A issued to Eleeza on Oct. 28, 1924 which discloses a test tube that is used to contain materials from a clinical and bacteriological laboratory, U.S. Pat. No. 2,460,641issued to Kleiner on Feb. 1, 1949 which discloses blood collecting apparatus that utilizes a vacuum mechanism to assist with drawing blood, U.S. Pat. No. 7,632,315B2 issued to Egilsson on Dec. 15, 2009 which discloses a method for creating a vacuum chamber, and U.S. Pat. No. 1,0723,538B2 issued to Reid on Jul. 28, 2020 which discloses a method for vacuum-insulating materials.

Advantageously, the test tube element contains one or multiple additives to alter or improve the general quality or to counteract undesirable properties in the blood sample in a manner that is responsive to the contemplated blood test. Disclosures of the use of additives, which are incorporated herein by reference in their entirety, include U.S. Pat. No. 5,320,812 issued to Harper on Jun. 14, 1994 which discloses a blood collection system that contains a clot activating polyelectrolyte complex, U.S. Pat. No. 5,344,611 issued to Wang on Sep. 17, 2013 which discloses a specimen collection container that assists with the stabilization of blood pH during sample storage, and U.S. Pat. No. 5,326,535 issued to Vogler on Jul. 5, 1994 which discloses a tube that has an interior coating of unitarily immobilized clotting activator. An additive comprises, for example, sodium fluoride, sodium heparin, EDTA, sodium citrate, heparin, gel, ACD, SPS, as well as all other additives. Examples of test tubes containing additives, the teachings of which are incorporated herein by reference, include the BD Vacutainer Heparin Tubes and the BD Vacutainer Citrate Tubes.

In an alternative embodiment, the test tube element is empty. Alternatively, the test tube element need not include a vacuum. Multiple blood collection apparatuses may be produced to accommodate different test tube element dimensions and volumes (e.g., 1 mL, 1.8 mL, 2 mL, 2.5 mL, 2.7 mL, 3 mL, 3.5 mL, 4 mL, 4.5 mL, 5 mL, 6 mL, 7 mL, 8 mL, 8.5 mL, 9 mL, 9.5 mL, 10 mL).

In chemistry, reactions that change temperature can be defined as either endothermic or exothermic. An endothermic reaction absorbs heat from its surroundings and an exothermic reaction releases heat into its surroundings. Heat is absorbed from the surroundings when chemical bonds are broken and heat is released into the surroundings when chemical bonds are formed. The amount of heat absorbed or released by a chemical reaction can be calculated using the equation q=mc ΔT, where q is the amount of heat energy, c is the specific heat capacity of the solution, m is the mass of the solution, and ΔT is the temperature change. The change in temperature (ΔT) is calculated by subtracting the initial temperature of the solution from the final temperature of the solution. The specific heat capacity of reagents and reactions are standard. The temperature change can be manipulated by altering the mass of the reactants.

Advantageously and innovatively, in the various embodiments disclosed herein, the blood collection apparatus comprises a heat transfer element that includes at least two reagents to initiate a chemical reaction (e.g., an endothermic reaction absorbing heat, or an exothermic reaction releasing heat). The heat transfer element, encapsulating the test tube element, absorbs heat from, or release heat to, the blood in the test tube element as the blood is drawn from the patient.

The amount of each reagent is responsive to the amount of time the reaction is intended to stay at a certain temperature. Advantageously, the amount of each reagent is chosen based on the desired temperature of the reaction and the desired heat transfer of the heat transfer element. Disclosures of the use of an endothermic reaction for heat transfer, which are incorporated herein by reference in their entirety, include U.S. Patent Application Pub. No. US2015/0253057A1 issued to Leavitt on Sep. 1, 2015 which discloses a cooling agent for cold packs and beverage containers. The cooling agent includes compounds such as potassium, nitrogen, ammonium phosphate, diammonium phosphate, ammonium polyphosphate, ammonium pyrophosphate, and ammonium metaphosphate, U.S. Patent Application Pub. No. US2017/0122645A1 to Berardino on May 4, 2017 which discloses an instant cold pack apparatus that utilizes ammonium nitrate, urea, ammonium formate, U.S. Patent Application Pub. No. US2010/0251731A1 issued to Bergida on Oct. 7, 2010 which discloses a self-chilling beverage can that utilizes an endothermic reaction and other chemicals to perform an endothermic reaction, and U.S. Pat. No. 6,438,965B1 issued to Liao on Aug. 27, 2002 which discloses an instant cold pack that comprises a water bag and a coolant consisting of ammonium, nitrate sodium carboxyl methyl cellulose, and sodium chloride.

The heat transfer element contains at least two reagents to produce an endothermic reaction or an exothermic reaction. In an endothermic reaction, the heat transfer element will absorb heat from the blood contained in the test tube element. Alternatively, in an exothermic reaction, the blood contained in the test tube will absorb heat from the heat transfer element. In some embodiments, the reagents mix, freeze, thaw, and refreeze to prolong the amount of time the system remains at a desired temperature. In some embodiments, the heat transfer element contains a substance or mechanism that causes a temperature change or allows for a temperature change, including a phase change material, wax, or other material that retains a desired temperature.

Additionally or alternatively, the heat transfer element of the blood collection apparatus stores a heat transfer agent, e.g., a non-reagent based cooling or heating element using, for example, ice, dry ice, cooling or heating gel that may promote maintaining a desired temperature. The term “heat transfer agent” shall mean a substance or mixture of substances that retain cold or hot temperatures. The heat transfer agent may be in any form of matter. A heat transfer agent herein comprises, for example, polymers, polymers combined with water, water, aerogel, and phase change material. Certain embodiments of a blood collection apparatus utilizing a heat transfer agent would require bringing the blood collection apparatus to a desired temperature prior to its use. For example, in using water as the heat transfer agent, the blood collection apparatus would need to be cooled or frozen and maintained at that temperature prior to its use.

In some embodiments, the heat transfer element stores a heat transfer agent that maintains a certain temperature when cooled with a freezer or other form of cooling mechanism. Alternatively, the heat transfer element contains one or more partitions housing water or another substance. The partition of the heat transfer element may be a vacuum. The heat transfer element encapsulating the test tube element may be a chamber of the test tube element.

The blood collection apparatus comprising a heat transfer element encapsulating the test tube element and storing at least two reagents is thus capable of initiating an endothermic reaction contemporaneously with the extraction of the blood from the patient. The endothermic reaction is described by an equation q=mcΔT, wherein “q” is a symbol for heat energy, “m” is a symbol for mass, “c” is a symbol for specific heat, and “ΔT” is a symbol for change in temperature.

Alternatively, the heat transfer element stores at least two reagents capable of initiating an exothermic reaction contemporaneously with the extraction of the blood from the patient. The exothermic reaction is described by an equation q=mcΔT, wherein “q” is a symbol for heat energy, “m” is a symbol for mass, “c” is a symbol for specific heat, and “ΔT” is a symbol for change in temperature.

The heat transfer element further comprises a fracturable element that separates and prevents the reagents from prematurely initiating the chemical reaction. When the fracturable element is fractured with the application of a fracture force, then at least two reagents initiate the endothermic or exothermic reaction. The user of the blood collection apparatus applies a fracture force to the fracture element. Examples of two reagents are ammonium nitrate and water. A reagent may be any chemicals, compounds, or materials that when mixed produce the desired endothermic or exothermic reaction.

Disclosures of the use of a fracturable element, which are incorporated herein by reference in their entirety, include U.S. Patent Application Pub. No. US2002/0012563A1 issued to May on Nov. 4, 2003 which discloses a rupturable membrane between two chambers that fractures when pressure is applied to it, U.S. Pat. No. 8,550,737B2 issued to Ruiz on Oct. 8, 2013 which discloses an applicator for dispensing adhesive or sealant material. It utilizes a sharp cutter within the apparatus designed to break a frangible seal on a container containing the adhesive, U.S. Pat. No. 1,0017,316B2 issued to May on Jul. 7, 2018 which discloses a container assembly that has two containers and the second container is rupturable by manipulation through the first container, and U.S. Pat. No. 5,879,635A issued to Nason on Mar. 9, 1999 which discloses a reagent dispenser that utilizes a deforming method in order to have two reagents mix. Examples of the commercial implementation of fracturable elements, the teachings of which are incorporated herein by reference, include Dermabond by Johnson and Johnson and Nozin antiseptic.

The fracturable element is composed of a material with a fracture strength that is responsive to the fracture of the fracturable element with a user's application of a fracture force. The fracturable element is composed of a brittle material such as chalk, glass, ceramic, or graphite. In some embodiments, the material covering the fracturable element is flexible to allow for a fracture force to be applied to the fracturable element. Alternatively, the material covering the fracturable element utilizes a button mechanism to apply the fracture force to the fracturable element. Disclosure of the use of a button mechanism, which is incorporated herein by reference in its entirety, includes U.S. Pat. No. 6765164B2 issued to Hyun-Mu Lee on Jul. 20, 2004 which discloses a push button seated in the seat depression so as to be movable by a predetermined distance.

The fracturable element can be composed of any other fracturable material. In an alternative embodiment, the fracturable element fractures by dissolving, melting, crumbling, collapsing, or splitting with applied force.

Advantageously, a colorant may be used to indicate the fracture of the fracturable element. The colorant will be activated upon mixture of the reagents separated by the fracturable element. In some embodiments, each of the reagents separated by the fracturable element will contain a colorant and the two colorants mix to form a distinct color. For example, one reagent may contain a yellow colorant and a second reagent may contain a blue colorant, and mixing of the two reagents following fracture of the fracturable element creates a green mixture. An indicator of the fracture of the fracturable element helps the user determine if the fracturable element has been effectively fractured. The indicator also allows the user to know if the fracturable element was fractured in transport or at a time prior to its intended use.

In an alternative embodiment, the fracturable element is an ampoule. Disclosures of the use of an ampoule, which are incorporated herein by reference in their entirety, include U.S. Pat. No. 5,379,898A issued to Joulia on Jan. 10, 1995 which discloses a self-breakable ampoule designed for easy flow of the product contained in the ampoule.

The ampoule of the blood collection apparatus contains one or more reagents. The ampoule is located at any location within the heat transfer element. The ampoule is broken to release its contents into the surrounding reagent or reagents. The ampoule is broken by a force applied on the side of the blood collection apparatus or applied to the end of the blood collection apparatus opposite the test tube septum. Alternatively, the ampoule is broken by shaking of the blood collection apparatus or by a material within the heat transfer element. Optionally, the heat transfer element may contain multiple ampoules which need not be fractured simultaneously or contemporaneously. Such a multiple ampoule use in the heat transfer element would enable revitalizing the endothermic or exothermic reaction at a subsequently advantageous moment.

Advantageously, certain embodiments of a blood collection apparatus further comprise a heat transfer element whose perimeter (i.e., outside wall, exterior surface, exterior material rather than its inner wall, interior surface, interior material) includes insulation beyond that which may be provided by the inner wall, interior surface, interior material. The insulation substantially helps retain the increased or decreased temperature within the heat transfer element and the test tube element.

Since insulation in the inner wall, interior surface, or interior material of the heat transfer element is at cross purposes with the object of the heat transfer element (i.e., transfer/exchange heat to or from the test tube element), advantageously the material of the inner wall of the heat transfer element ought to promote a transfer/exchange of heat (i.e., a conducting material). In such embodiments the material of the perimeter would be responsibly different from the material of inner wall/surface of the heat transfer element. In other words, a perimeter of the heat transfer element including insulation ought to be understood as being materially different in its inhibiting heat transfer properties than the heat transfer properties of the inner wall, interior surface, or interior material of the heat transfer element.

The insulation also prevents a user's hands from being exposed to or affecting the varying temperatures of the device. The insulation can be sprayed or painted on, made a part of, or otherwise incorporated into, the perimeter of the heat transfer element.

In another alternate embodiment, the blood collection apparatus further comprises an insulation element that encapsulates the heat transfer element. The insulation element utilizes or is composed of an insulator comprising, for example, a vacuum, rubber, plastic, acrylic, fiberglass, polyurethane, styrofoam, cork, asbestos, thermoplastic, cellulose, polystyrene, wool, or any other thermal insulating material. The insulation element may include a combination of or layers of a non-insulating material and/or an insulating material.

Alternative or additionally, the insulation, insulator, and insulation element may comprise a light filter to protect the blood from the effects of light external to the blood collection apparatus.

The blood collection apparatus has a test tube septum. Advantageously, the test tube septum comprises an indicator of a heat transfer capability of the blood collection apparatus. The test tube septum is comprised of any material of any composition that covers or seals the opening of a test tube element and which may be puncturable. Advantageously, the test tube septum comprises an insulating material. The test tube septum may be any color or size.

Conventionally, test tubes are sealed with a test tube septum and often have a specific additive placed in the tube with the test tube septum color indicating the additive. For example, a blue-top tube is a 5 ml test tube containing sodium citrate as an anticoagulant. Advantageously, the conventional color scheme implemented in a test tube septum is combined with a designation indicating the endothermic or exothermic capability of the blood collection apparatus. For example, in an endothermic capable blood collection apparatus, in addition to the appropriate additive color designation, a snowflake image is added. Alternatively, the heat transfer designation may be any color, combination of colors (e.g., stripping), and images (e.g., a snowflake, fire, spark, thermometer), logo, and/or suitable design.

Advantageously, the various embodiments disclosed herein comprise a blood collection apparatus that may be used in conjunction with a specifically adapted device or general purpose devices such as a vacutainer hub, vacutainer holder, syringe, and other blood draw devices. Such general purpose devices conventionally comprise a test tube needle holder, test tube needle, butterfly, and butterfly needle. The size of conventional vacutainer hubs, vacutainer needle holders, and syringes need not need to be changed to accommodate the blood collection apparatus. In the case of a specifically adapted device or a special purpose device, such devices are responsive to the dimensional and heat transfer requirements of the blood collection apparatus.

The blood collection apparatus is also specially adapted to be used in conjunction with g centrifuges and blood processing equipment. Conventional centrifuges and blood processing equipment need not be altered to accommodate the blood collection apparatus. Examples of commercially available test tube needle holders include the BD Vacutainer One-Use Holder, The BD Vacutainer One-Use Needle Holder, and the Vacutainer Hub, the teachings of which are incorporated herein by reference.

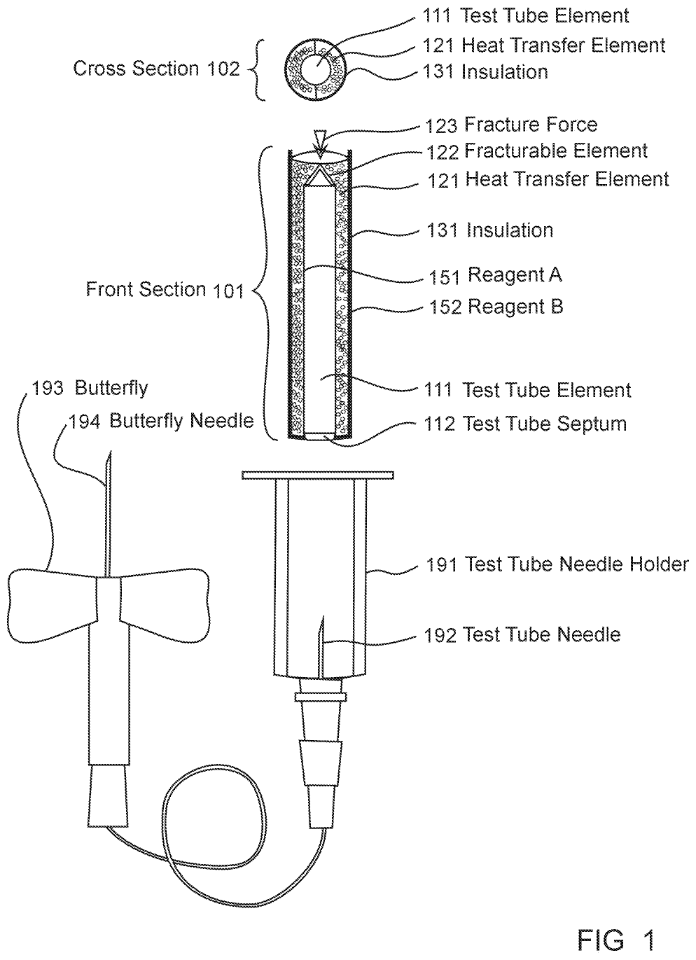

FIG. 1 is a front section view 101 and a cross section view 102 of a blood collection apparatus embodiment comprising a test tube element 111 and test tube septum 112. The test tube element 111 is vacuum sealed to assist with drawing blood into the tube. Advantageously, the test tube element 111 contains one or multiple additives to alter or improve the general quality or to counteract undesirable properties in the blood sample in a manner that is responsive to the contemplated blood test.

The blood collection apparatus of FIG. 1 further comprises a heat transfer element 121. The heat transfer element 121 contains one or more reagents (e.g., reagent A 151 and reagent B 152) to produce an endothermic reaction or an exothermic reaction. In an endothermic reaction, the heat transfer element 121 will absorb heat from the blood contained in the test tube element 111. Alternatively, in an exothermic reaction, the blood contained in the test tube 111 will absorb heat from the heat transfer element 121. The heat transfer element 121 further comprises a fracturable element 122 that is fractured with the application of a fracture force 123 enabling the at least two reagents to initiate the endothermic or exothermic reaction. The user of the blood collection apparatus applies a fracture force 123 to the fracture element 122. Fracture of the fracture element 122 allows at least two reagents (e.g., reagent A 151 and reagent B 152) to mix, initiating the heat transfer process. Examples of two reagents are ammonium nitrate and water.

The heat transfer element further comprises an insulation of its perimeter 131 that maintains the increased or decreased temperature within the heat transfer element 121 and the test tube element 111. Advantageously, the insulation 131 also prevents a user's hands from being exposed to or affecting the varying temperatures of the device. The insulation 131 can be sprayed, painted, or otherwise incorporated into the outer wall of the heat transfer element 121. The insulation capability of the perimeter of the heat transfer element is shown in FIG. 1 as the bold line.

In the embodiment shown in FIG. 1, the fracturable element 122 is located at the bottom of the blood collection apparatus opposite the test tube septum 112 which is located on the top of the blood collection apparatus (the blood collection apparatus is illustrated upside down). Unintentional application of a fracture force 123 is inhibited by, for example, an extension of the wall of the insulation element 131. The fracturable element 122 separates the reagents in the heat transfer element 121.

For perspective, FIG. 1 also depicts a test tube needle holder 191, test tube needle 192, butterfly 193, and butterfly needle 194. The size of conventional vacutainer hubs, vacutainer needle holders, and syringes need not need to be changed to accommodate the blood collection apparatus. Conventional centrifuges and blood processing equipment need not be altered to accommodate the blood collection apparatus.

In an exemplary embodiment relating to FIG. 1 and the disclosure herein, a blood collection apparatus comprises: (i) a test tube element comprising a vacuum facilitating an extraction of blood from a patient, and further comprising a test tube septum, the test tube septum comprising an indicator of an endothermic capability of the blood collection apparatus; (ii) a heat transfer element encapsulating the test tube element and storing at least two reagents capable of initiating an endothermic reaction contemporaneously with the extraction of the blood from the patient, the endothermic reaction described by equation q=mcΔT, wherein “q” is a symbol for heat energy, “m” is a symbol for mass, “c” is a symbol for specific heat, and “ΔT” is a symbol for change in temperature, said endothermic reaction absorbing heat from the blood in the test tube element, the heat transfer element further comprising a fracturable element that when fractured enables the at least two reagents to initiate the endothermic reaction; and (iii) wherein the heat transfer element includes insulation inhibiting a loss of a temperature change of the blood resulting from the endothermic reaction initiated by the at least two reagents, wherein the insulation does not interfere with a fracturing of the fracturable element, and wherein the combination of the heat transfer element and test tube element comprises a circumference suitable to be mated with a general purpose test tube needle holder.

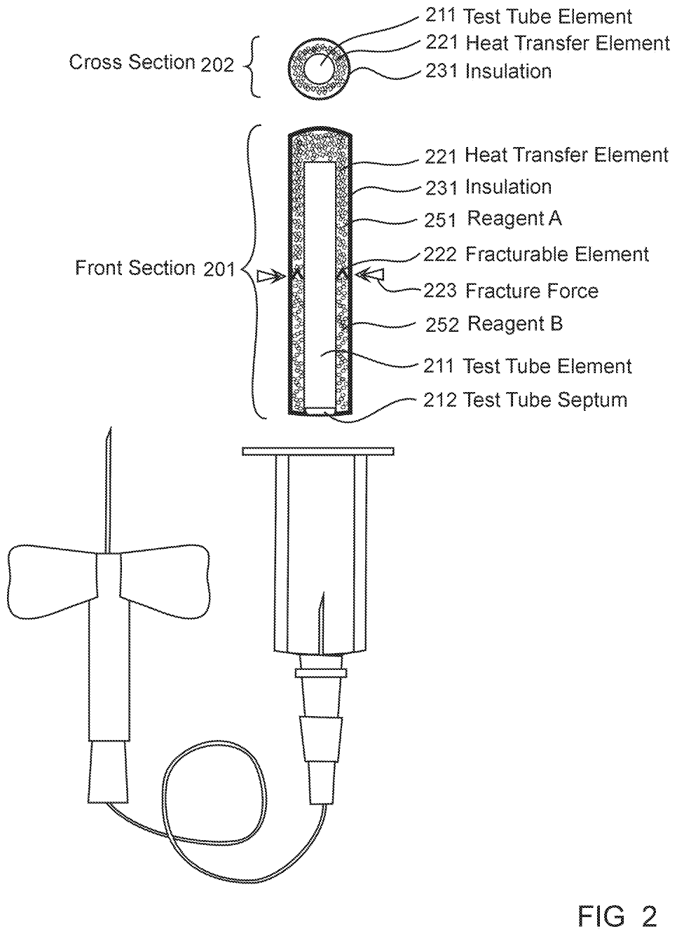

FIG. 2 is a front section view 201 and a cross section view 202 of a blood collection apparatus comprising a test tube element 211, a test tube septum 212, a heat transfer element 221, a fracturable element 222, a reagent A 251, and a reagent B 252. In this embodiment, a fracture force 223 is applied on the side of the blood collection apparatus to fracture the fracturable element 222.

In this embodiment, the heat transfer element 221 comprises insulation on the exterior perimeter 231 of the heat transfer element 221 , and is configured to accommodate the fracturable element 222 being located on the side of the blood collection apparatus. The fracturable element separates one or more reagents (e.g., reagent A 251 and reagent B 252). Similarly, the insulation 231 is configured to accommodate the fracturable element 222 being located on the side of the blood collection apparatus. The insulation 231 is flexible to allow for the fracture force 223 to fracture the fracturable element 222. Advantageously, the insulation 231 is a material that is both insulating and flexible, such as rubber, plastic, or any other flexible insulating material. Alternatively, the entirety of the insulation 231 need not be flexible, partial flexibility or a push button implementation for that portion of the heat transfer element 121 necessary to accommodate the application of the fracture force 223 may be implemented.

Reagent A 251 and reagent B 252 are configured to accommodate the fracturable element 222 being located on the side of the collection apparatus. The fracture force 223 is applied to the side of the blood collection apparatus to fracture the fracturable element 222, causing reagent A 251 and reagent B 252 to mix, initiating a reaction.

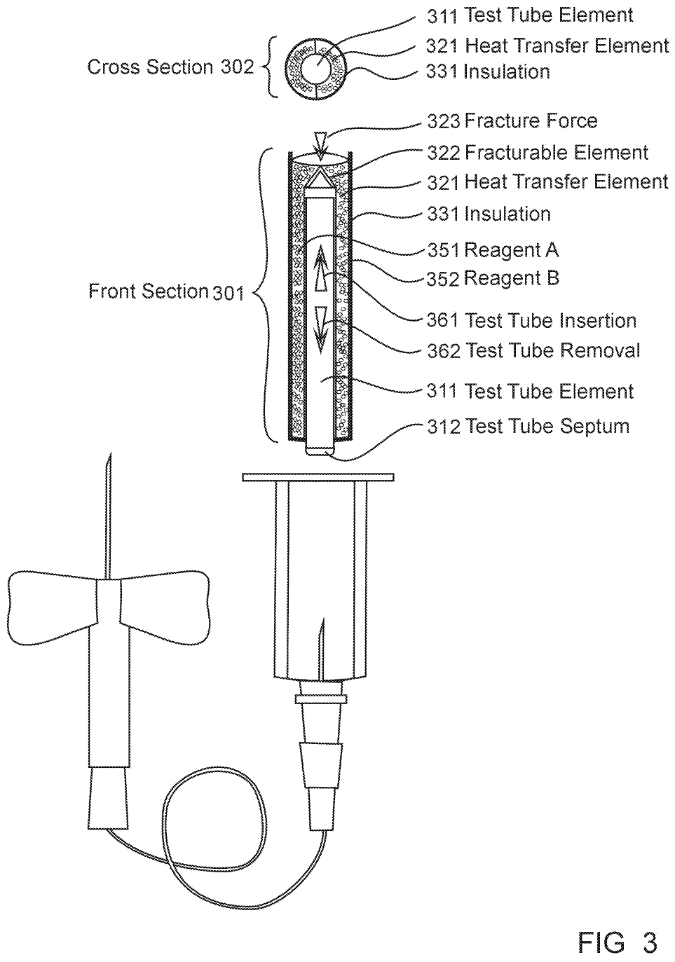

FIG. 3 is a front section view 301 and a cross section view 302 of a blood collection apparatus comprising a test tube element that is removable from the blood collection apparatus. In this embodiment, the test tube element 311 whether a general purpose or specially adapted test tube element is independent from the heat transfer element 321. The test tube element 311 is removable from the blood collection apparatus at any point in the blood collection and analysis process. Optionally, the test tube element 311 may be kept within the blood collection apparatus throughout the blood collection and analysis process.

The test tube element 311 is inserted by, for example, a pushing or sliding force 361 into the heat transfer element 321. Similarly, the test tube element 311 is removed by, for example, a pulling or sliding force 362 away from the heat transfer element 321. In this embodiment, the structures of the test tube septum 312, heat transfer element 321, and insulation 331 are configured to enable the removal of the test tube element 311 from the heat transfer element 321.

As in FIG. 1, in the embodiment of FIG. 3, the fracturable element 322 is located on the top of the blood collection apparatus. However, both the fracturable element 322 and the heat transfer element 321 are modified as shown to compensate for the removable of the test tube element 311. The fracturable element 322 is fractured by a fracture force 323 to cause the reaction between the reagent A 351 and the reagent B 352.

In an exemplary embodiment relating to FIG. 3 and the disclosure herein, a blood collection apparatus comprises: (i) a test tube element comprising a vacuum facilitating an extraction of blood from a patient, and further comprising a test tube septum, the test tube septum comprising an indicator of an endothermic capability of the blood collection apparatus; (ii) a heat transfer element encapsulating the test tube element and storing at least two reagents capable of initiating an endothermic reaction contemporaneously with the extraction of the blood from the patient, said endothermic reaction absorbing heat from the blood in the test tube element, the heat transfer element further comprising a fracturable element that when fractured enables the at least two reagents to initiate the endothermic reaction; and (iii) wherein the heat transfer element comprises an insulation inhibiting a loss of a temperature change of the blood resulting from the endothermic reaction initiated by the at least two reagents, wherein the insulation does not interfere with a fracturing of the fracturable element, wherein the combination of the heat transfer element and the test tube element comprises a circumference suitable to be mated with a general purpose test tube needle holder, and wherein the test tube element is removable from the heat transfer element contemporaneously with an initiation of a blood test.

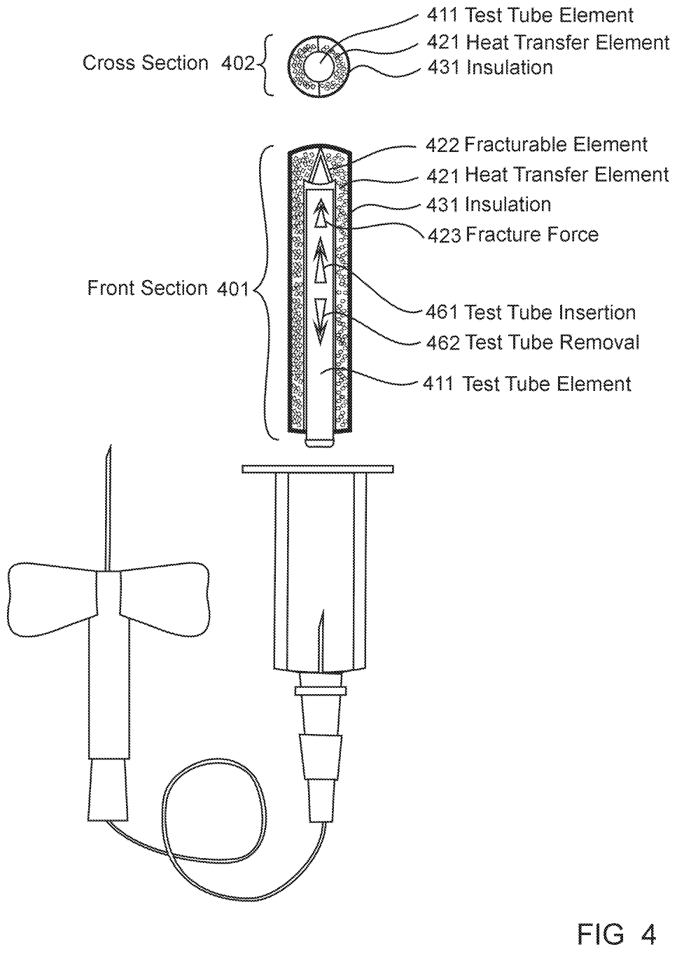

FIG. 4 is a front section view 401 and a cross section view 402 of a blood collection apparatus comprising a test tube element that is removable from the blood collection apparatus. In the embodiment of FIG. 4, a fracture force 423 is applied to the modified fracturable element 422 by the insertion of the test tube element 411 into the blood collection apparatus. Advantageously, a general purpose test tube element (e.g. a test tube) or a specially adapted test tube element that are removable from the blood collection apparatus provide the necessary element to apply a fracture force 423 to the fracturable element 422.

The test tube element 411 is inserted by, for example, a pushing or sliding force 461 into the heat transfer element 421. Similarly, the test tube element 411 is removed by, for example, a pulling or sliding force 462 away from the heat transfer element 421.

In this embodiment, the structures of the test tube septum 412, heat transfer element 421, and insulation 431 are configured to enable the removal of the test tube element 411 from the heat transfer element 421. Additionally, in this embodiment, the structure of the heat transfer element 421 and insulation 431 are compatible with a removable test tube element 411 and the use of the test tube element 411 to fracture the fracturable element 422.

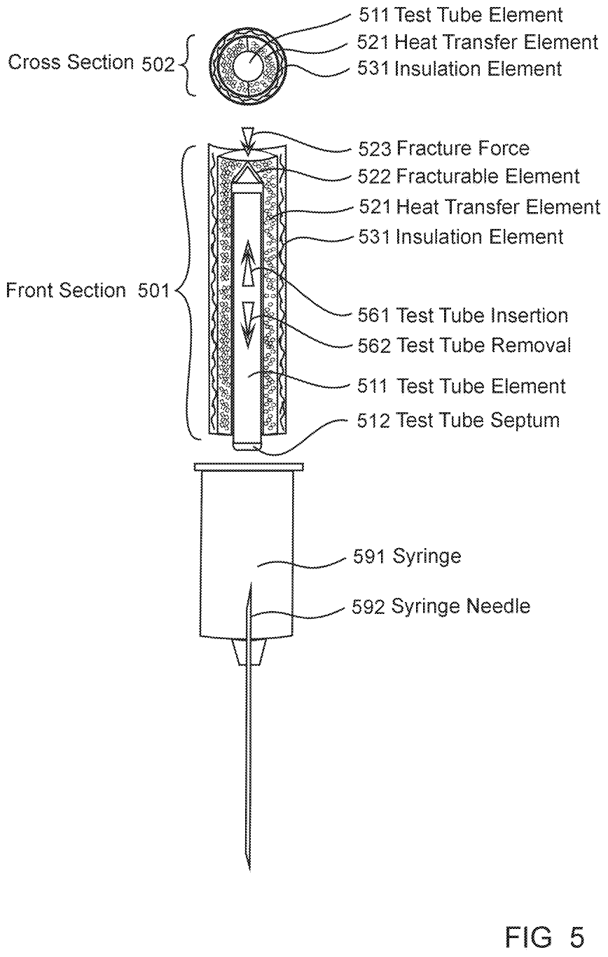

FIG. 5 is a front section view 501 and a cross section view 502 of a blood collection apparatus comprising a test tube element 511 that is removable from the blood collection apparatus. However, by contrast to the embodiment of FIG. 4, and similar to the embodiment of FIG. 1, the embodiment of FIG. 5 comprises a fracturable element 522 that is fractured with the application of a fracture force 523 external to blood collection apparatus which enables at least two reagents to initiate an endothermic or an exothermic reaction.

Advantageously, the embodiment of FIG. 5 further comprises an insulation element 531 that encapsulated the heat transfer element 521 and that provides an enhanced and more substantial level of insulation than what may or may not be provided by the perimeter of the heat transfer element 521. The insulation element 531 utilizes or is composed of an insulator comprising, for example, a vacuum, rubber, plastic, acrylic, fiberglass, polyurethane, styrofoam, cork, asbestos, thermoplastic, cellulose, polystyrene, wool, or any other thermal insulating material. The insulation element 531 may include a combination of or layers of a non-insulating material and/or an insulating material.

The structure of the insulation element 531 and the heat transfer element 521 while configured responsive to the fracturable element 522, may, although not necessarily, also accommodate a removable test tube element 511. On top of the fracturable element 522, the insulation element 531 may be composed of a flexible thermal insulation material. Alternatively, the required fracture force 523 may be applied to the fracturable element 522 by, for example, a flexible thermal insulation material such as rubber acting as a push button mechanism.

The test tube element 511 is inserted by, for example, a pushing or sliding force 561 into the heat transfer element 521. Similarly, the test tube element 511 is removed by, for example, a pulling or sliding force 562 away from the heat transfer element 521. In this embodiment, the structures of the test tube septum 512, heat transfer element 521, and insulation element 531 are configured to enable the removal of the test tube element 511 from the heat transfer element 521.

The embodiment of FIG. 5 is illustrated in combination with the use of a general purpose syringe 591 and syringe needle 592. The syringe 591 and syringe needle 592 draw blood from the patient without the use of the butterfly needle or butterfly. Advantageously, in the embodiment of FIG. 5, the blood collection apparatus is dimensionally compatible with a general purpose syringe 591. In such an embodiment, a general purpose syringe need not be changed to accommodate the blood collection apparatus. Alternatively, a blood collection apparatus is mated with a special purpose syringe that specifically accommodates the dimensional and heat transfer requirements of the test tube element 511, heat transfer element 531, and insulation element 531.

In an exemplary embodiment relating to FIG. 5 and the disclosure herein, a blood collection apparatus comprises: (i) a test tube element comprising a vacuum facilitating an extraction of blood from a patient, and further comprising a test tube septum; (ii) a heat transfer element encapsulating the test tube element and storing at least two reagents capable of initiating a chemical reaction contemporaneously with the extraction of the blood from the patient, the chemical reaction exchanging heat with the blood, the heat transfer element further comprising a fracturable element that when fractured enables the at least two reagents to initiate the chemical reaction; and (iii) an insulation element encapsulating the heat transfer element, the insulation element inhibiting a loss of a temperature change of the blood resulting from the chemical reaction initiated by the at least two reagents; wherein the insulation element does not interfere with a fracturing of the fracturable element; wherein the chemical reaction is an endothermic reaction described by an equation q=mcΔT, wherein “q” is a symbol for heat energy, “m” is a symbol for mass, “c” is a symbol for specific heat, and ΔT is a symbol for change in temperature, wherein the endothermic reaction absorbs heat from the blood; wherein the insulation element does not interfere with the test tube element being removed from the heat transfer element contemporaneously with an initiation of a blood test; wherein the insulation element includes an insulator selected from the group consisting of a vacuum, rubber, plastic, acrylic, fiberglass, polyurethane, styrofoam, cork, asbestos, thermoplastic, cellulose, polystyrene, wool, and thermal insulating material; wherein the chemical reaction is an endothermic reaction, wherein the test tube septum comprises an indicator of an endothermic capability of the blood collection apparatus; and wherein the chemical reaction is an endothermic reaction, wherein the at least two reagents are water and ammonium nitrate; and wherein the blood collection apparatus comprises a circumference suitable to be mated with a general purpose test tube needle holder and/or a general purpose syringe;.

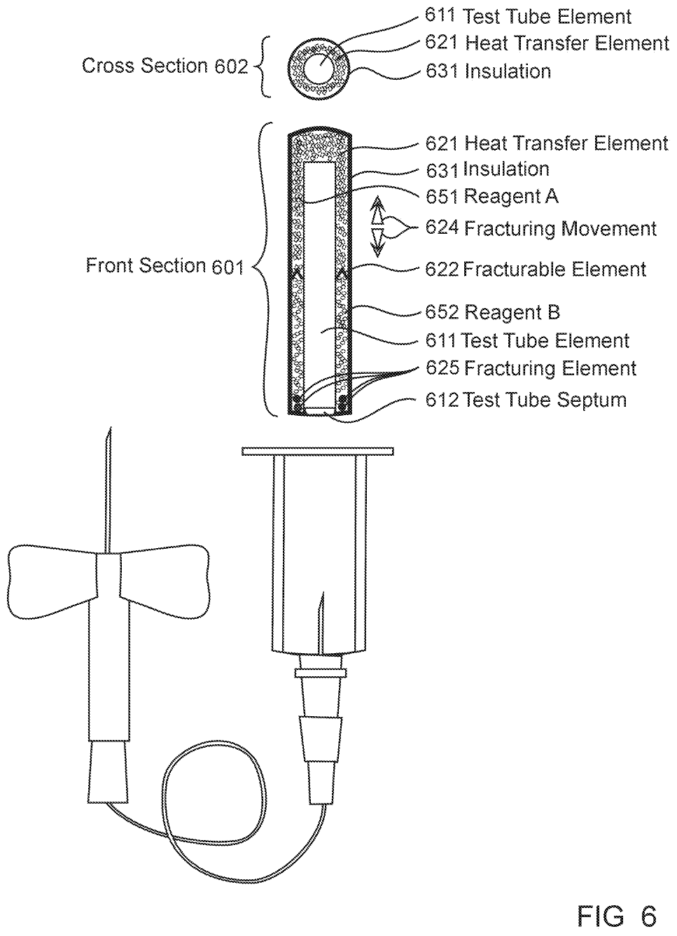

FIG. 6 is a front section view 601 and a cross section view 602 of a blood collection apparatus comprising a test tube element 611, test tube septum 612, and heat transfer element 621 comprising insulation 631 wherein a fracturing movement 624 is applied bidirectionally to cause a fracturing element 625 to fracture the fracturable element 622.

The fracturable element 622 is located on the side of the blood collection apparatus. The fracturing element 625 may, although not necessarily, be spherical in shape and include multiple spherical elements. The fracturable element 625 may be located in any location within the heat transfer element 621. Advantageously, the fracturing element 625 serves to enable mixing the reagents and may be, as are other features and elements disclosed herein, used in combination with other fracturable elements detailed herein.

The heat transfer element 621 is structured to accommodate the fracturing element 622, located on the side of the blood collection apparatus, and the fracturing movement 624 to the enable the fracturing element 625 to fracture the fracturable element 622 and initiate the chemical reaction between the one or more reagents (e.g., reagent A 651 and reagent B 652).

The heat transfer element 621 and insulation 631 are configured to accommodate the fracturable element 622 being located on the side of the blood collection apparatus.

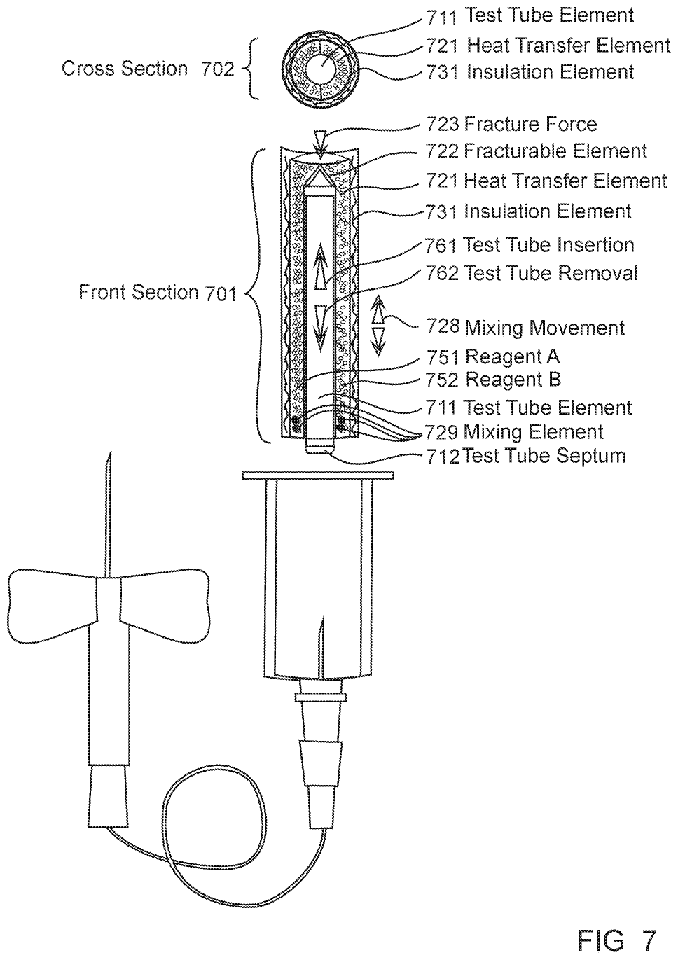

FIG. 7 is a front section view 701 and a cross section view 702 of a blood collection apparatus comprising a test tube element 711, test tube septum 712, heat transfer element 721, and an insulation element 731 wherein a fracturing force 723 is applied to fracture a fracturable element 722. A bidirectional mixing movement 728 enables a mixing element 729 to promote the mixing of the at least two reagents (e.g., reagent A 751 and reagent B 752) and initiate the chemical reaction.

Similar to the embodiment of FIG. 1, in the embodiment shown in FIG. 7, the fracturable element 722 is located at the bottom of the blood collection apparatus opposite the test tube septum 712 which is located on the top of the blood collection apparatus (the blood collection apparatus is illustrated upside down). In this embodiment, unintentional application of a fracture force 723 is inhibited by, for example, a curvature to that portion of the insulation element 731 to inhibit an unintended fracture of the fracturable element 722. The fracturable element 722 separates the reagents in the heat transfer element 721 until the fracturing of the fracturable element 722.

Similar to the embodiment of FIG. 3, advantageously in the embodiment shown in FIG. 7, the blood collection apparatus comprises a test tube element 711 that is removable from the blood collection apparatus. The test tube element 711 is inserted by, for example, a pushing or sliding force 761 into the heat transfer element 721. Similarly, the test tube element 711 is removed by, for example, a pulling or sliding force 762 away from the heat transfer element 721. In this embodiment, the structures of the test tube septum 712, heat transfer element 721, and insulation element 731 are configured to enable the removal of the test tube element 711 from the heat transfer element 721.

The test tube element 711, whether a general purpose or specially adapted test tube element, is independent from the heat transfer element 721. The test tube element 711 is removable from the blood collection apparatus at any point in the blood collection and analysis process. Optionally, the test tube element 711 may be kept within the blood collection apparatus throughout the blood collection and analysis process.

Advantageously, as in the embodiment of FIG. 5, the embodiment of FIG. 7 further comprises an insulation element 731 encapsulating the heat transfer element 721.

Importantly, FIG. 7 is drawn to illustrate the many potential advantageous synergistic combinations of elements that are available herein. Clearly, the particular elements, features, structures, or characteristics may be combined in a manner suitable to a particular requirement.

In an exemplary embodiment relating to FIG. 7 and the disclosure herein, a blood collection apparatus comprises: (i) a test tube element comprising a vacuum facilitating an extraction of blood from a patient, and further comprising a test tube septum; (ii) a heat transfer element encapsulating the test tube element and storing at least two reagents capable of initiating a chemical reaction contemporaneously with the extraction of the blood from the patient, the chemical reaction exchanging heat with the blood, the heat transfer element further comprising a fracturable element that when fractured enables the at least two reagents to initiate the chemical reaction; and (iii) an insulation element encapsulating the heat transfer element, the insulation element inhibiting a loss of a temperature change of the blood resulting from the chemical reaction initiated by the at least two reagents; wherein the insulation element does not interfere with a fracturing of the fracturable element; wherein the heat transfer element encapsulating the test tube element further comprises a reagents mixing element; wherein the insulation element does not interfere with the test tube element being removed from the heat transfer element contemporaneously with an initiation of a blood test; and wherein the blood collection apparatus comprises a circumference suitable to be mated with a general purpose test tube needle holder.

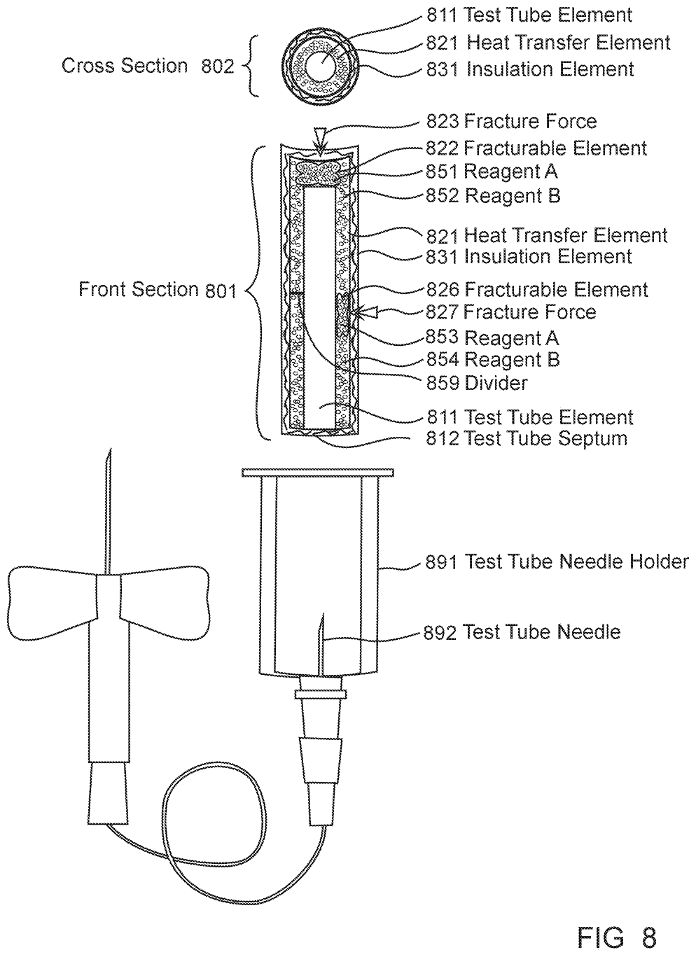

FIG. 8 is a front section view 801 and a cross section view 802 of a blood collection apparatus comprising a test tube element 811, heat transfer element 821, and an insulation element 831. In this embodiment, the heat transfer element comprises a fracturable element that is a breakable ampoule 822 storing a least one reagent (e.g., reagent A 851). Outside of the ampoule 822, the heat transfer element 821 stores a least one other reagent (e.g., reagent B 852).

Similar to the embodiment of FIG. 7, in the embodiment shown in FIG. 8 the fracturable element (in this embodiment the ampoule 822) is located at the bottom of the blood collection apparatus opposite where the needle is inserted (the blood collection apparatus is illustrated upside down). In this embodiment, unintentional application of a fracture force 823 is inhibited by, for example, a curvature to that portion of the insulation element 831 to inhibit an unintended fracture of the ampoule 822. The ampoule 822 also separates the reagents (e.g., reagent A 851 and reagent B 852) in the heat transfer element 821 until the fracturing of the ampoule 822.

Optionally, the heat transfer element 821 is divided in at least two separately activated endothermic and exothermic zones by the use of for example, a second fracturable element (in this embodiment a second ampoule 826 also acting as a divider), and at least one heat transfer element divider 859. Within this zone created by the second fracturable element (in this embodiment a second ampoule 826) and the heat transfer element 821 divider 859, the ampoule 826 stores a least one reagent (e.g., in this illustration a second portion of reagent A 853), and the responsive zone of the heat transfer element 821 stores a least another reagent (e.g., in this illustration a second portion of reagent B 854).

Alternatively or advantageously, the second ampoule 826 contains the same reagent as is contained in the ampoule 822. The heat transfer element 821 is not divided and the divider 859 is absent. Following the reaction between reagent B 852 and the reagent housed in the ampoule 822, unused reagent B 852 remains. Fracturing of the second ampoule 822 causes a reaction between the reagent housed in ampoule 822 and the remaining reagent B, prolonging the heat transfer of the heat transfer element 821.

Alternative embodiments implementing multiple heat transfer element 821 zones are not limited to: (i) the first fracturable element (e.g. a first ampoule 822) and a second fracturable element (e.g., the second ampoule 826) storing the same reagent (e.g., reagent A) or equivalent proportions of the same reagent; (ii) the combination of reagents in each zone producing the same chemical process; (iii) the fracture of the first ampoule 822 and the fracture second ampoule 826 occurring simultaneously or contemporaneously; and (iv) both chemical processes being an endothermic reaction or exothermic reaction. Optionally, an embodiment would find advantageous to combine separate endothermic and exothermic reactions where, for example, an initial endothermic reaction is used to cool the blood and a subsequent exothermic reaction is used to warm the blood. In a non-simultaneous or non-contemporaneous initiation of the at least two chemical processes enabled by a responsive heat transfer element 821, a fracture force 823 would be applied to a first fracturable element 822 and at a subsequent time another fracture force 827 would be applied to a second fracturable element 826.

Advantageously, this embodiment further comprises an insulation element 831 that encapsulates the heat transfer element 821 and that provides an enhanced and more substantial level of insulation than what may or may not be provided by the perimeter of the heat transfer element 821. The insulation element 831 utilizes or is composed of an insulator comprising, for example, a vacuum, rubber, plastic, acrylic, fiberglass, polyurethane, styrofoam, cork, asbestos, thermoplastic, cellulose, polystyrene, wool, or any other thermal insulating material. The insulation element 831 may include a combination of or layers of a non-insulating material and/or an insulating material.

Optionally as is illustrated in FIG. 8, the insulation element 831 substantially completely encapsulates the heat transfer element 821 and the test tube element 811. In such an embodiment, the base of the blood collection apparatus that is inserted into, for example, the test tube needle 892 of the test tube needle holder 891 is responsive to the functional requirements of a test tube septum 812. In other words, the insulation materials utilized in at least the base would perform as is functionally required of a general purpose test tube septum.

In an exemplary embodiment relating to FIG. 8 and the disclosure herein, a blood collection apparatus comprises: (i) a test tube element comprising a vacuum facilitating an extraction of blood from a patient, and further comprising an indicator of an chemical process capability of the blood collection apparatus; (ii) a heat transfer element encapsulating the test tube element and including at least one breakable ampoule storing at least one reagent, the heat transfer element storing at least a second reagent that when combined with the first reagent is capable of initiating a chemical process contemporaneously with the extraction of the blood from the patient, the heat transfer element further comprising a mixing element whereby a bidirectional mixing movement enables the mixing element to promote the mixing of the at two or more reagents; and (iii) an insulation element substantially completely encapsulating the heat transfer element; wherein the insulation element does not interfere with a fracturing of the fracturable element; wherein a base of the blood collection apparatus is responsive to the functional requirements of a test tube septum; and wherein the combination of the insulation element, heat transfer element, and test tube element comprises a circumference suitable to be mated with a general purpose test tube needle holder. Optionally, in the exemplary embodiment relating to FIG. 8 and the disclosure herein, the heat transfer element further includes at least a second breakable ampoule storing at least a second portion of the one reagent, the heat transfer element storing at least a second portion of the second reagent that when combined with the second portion of the first reagent is capable of initiating a chemical process subsequently to an initial initiation of a chemical process within the heat transfer element.

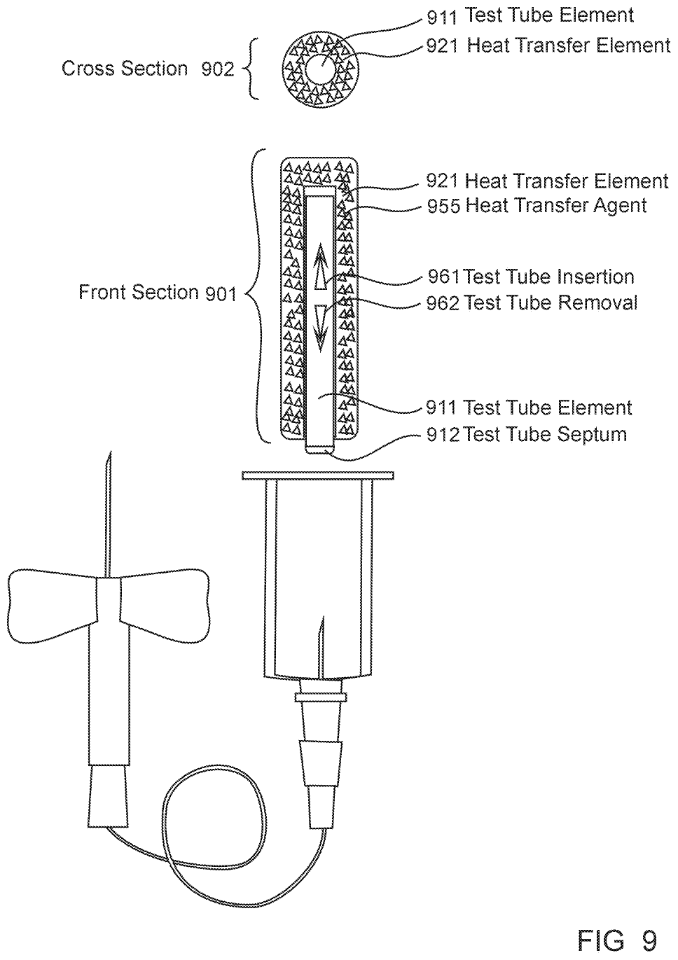

FIG. 9 is a front section view 901 and a cross section view 902 of a blood collection apparatus comprising a test tube element 911, test tube septum 912, and a heat transfer element 921. The heat transfer element 921 comprising a heat transfer agent 955.

As opposed to other embodiments, the embodiment of FIG. 9 does not include insulation (e.g., insulation 131 FIG. 1) or an insulation element (e.g., insulation element 531 FIG. 5). However, other embodiments may include such insulation and/or insulation element.

Similar to the embodiment of FIG. 7, advantageously in the embodiment shown in FIG. 9, the blood collection apparatus comprises a test tube element 911 that is removable from the blood collection apparatus. The test tube element 911 is inserted by, for example, a pushing or sliding force 961 into the heat transfer element 921. Similarly, the test tube element 911 is removed by, for example, a pulling or sliding force 962 away from the heat transfer element 921. In this embodiment, the structures of the test tube septum 912, heat transfer element 921, and insulation element 931 are configured to enable the removal of the test tube element 911 from the heat transfer element 921.

The use of a heat transfer agent 955 in the heat transfer element 921 in synergistic combination with a removable test tube element 911 has the advantage of a potential reusability of the portion of the blood collection apparatus comprising the heat transfer element 921 and the insulation element 931. In such an embodiment, for example, following an initial use and removal of the utilized test tube, at least the heat transfer element 921 and the insulation element 931 would be subjected to the required cooling or heating of the heat transfer element 921 and stored for subsequent utilization with a new test tube element 911.

Reference throughout this specification to “one embodiment” or “an embodiment” means that a particular element, feature, structure, or characteristic described in connection with the embodiment is included in at least one embodiment. Thus, appearances of the phrases “in one embodiment” or “in an embodiment” in various places throughout this specification are not necessarily all referring to the same embodiment.

The previous description is provided to enable any person skilled in the art to practice the various aspects described herein. Various modifications to these aspects will be readily apparent to those skilled in the art, and the generic principles defined herein may be applied to other aspects.

In an exemplary embodiment relating to FIG. 9 and the disclosure herein, a blood collection apparatus comprises: a test tube element comprising a vacuum facilitating an extraction of blood from a patient, and further comprising a test tube septum; and a heat transfer element encapsulating the test tube element and capable of initiating a heat transfer process contemporaneously with the extraction of the blood from the patient; wherein the heat transfer element encapsulating the test tube element stores a heat transfer agent for initiating the heat transfer process contemporaneously with the extraction of the blood from the patient; wherein a perimeter of the heat transfer element includes insulation inhibiting a loss of a temperature change of the blood resulting from the heat transfer process; wherein the test tube element is a general purpose test tube removable from the heat transfer element contemporaneously with an initiation of a blood test; and wherein the blood collection apparatus further comprises a circumference suitable to be mated with a general purpose test tube needle holder or a general purpose syringe.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

The teachings disclosed herein, directly and indirectly by, for example, incorporation, are intended to show a variety of inventive elements and features which are combined and may be combined to suit particular embodiments. While a function of incorporation is to provide additional detail explanation, the synergies among and between the various inventive elements is a significant feature of and object of incorporation. The incorporation by reference at a specific place within the specification is not intended to limit the extent to which the reference is incorporated, or the manner in which it may be integrated. Where a teaching may be deemed to be at cross purposes, or otherwise incompatible, with some other teaching, it ought to be understood as a possible alternative to be utilized as a particular preferred embodiment may require.

While elements of the inventions have been detailed in conjunction with specific embodiments thereof, it is evident that many alternative permutations in the combination elements and features are possible, and additional modifications and variations are possible and will be apparent to those skilled in the art in light of the foregoing descriptions. Accordingly, it is intended to embrace all such permutations, alternatives, modifications, variations, and combinations as fall within the spirit and broad scope of the specification. The teachings that have been cited and incorporated herein are offered by way of example, and not limitation, of the underlying foundation of knowledge and skill that is available to a person of ordinary skill in the art. Many of the features, components, and methods found in the art may be incorporated, as suggested herein, in a preferred embodiment; and since other modifications and changes varied to fit particular requirements and environments will be apparent to those skilled in the art, the inventions are not limited to the embodiments set forth or suggested herein. It is to be understood that the inventions are not limited thereby. It is also to be understood that the specific details shown are merely illustrative, and that the inventions may be carried out in other ways without departing from the broad spirit and scope of the specification.

What is claimed is:

1. A blood collection apparatus comprising:
a test tube element comprising a vacuum facilitating an extraction of blood from a patient, and further comprising a test tube septum;
a heat transfer element encapsulating the test tube element and storing at least two reagents capable of initiating a chemical reaction contemporaneously with the extraction of the blood from the patient, the chemical reaction exchanging heat with the blood, the heat transfer element further comprising a fracturable element that when fractured enables the at least two reagents to initiate the chemical reaction; and
an insulation element encapsulating the heat transfer element, the insulation element inhibiting a loss of a temperature change of the blood resulting from the chemical reaction initiated by the at least two reagents, wherein the insulation element does not interfere with a fracturing of the fracturable element, wherein the blood collection apparatus further comprises a circumference suitable to be mated with a general purpose test tube needle holder.

2. The blood collection apparatus of claim 1, wherein the chemical reaction is an endothermic reaction described by an equation q=mcΔT, wherein “q” is a symbol for heat energy, “m” is a symbol for mass, “c” is a symbol for specific heat, and ΔT is a symbol for change in temperature, and wherein the endothermic reaction absorbs heat from the blood.

3. The blood collection apparatus of claim 1, wherein the insulation element does not interfere with the test tube element being removed from the heat transfer element.

4. The blood collection apparatus of claim 1, wherein the chemical reaction is an endothermic reaction, and wherein the test tube septum comprises an indicator of an endothermic capability of the blood collection apparatus.

5. The blood collection apparatus of claim 1, wherein the chemical reaction is an endothermic reaction, and wherein the at least two reagents are water and ammonium nitrate.

6. The blood collection apparatus of claim 1, wherein the heat transfer element encapsulating the test tube element further comprises a reagents mixing element.

7. The blood collection apparatus of claim 1, wherein the heat transfer element further comprises an ampoule storing at least one of the at least two at least two reagents.

8. A blood collection apparatus comprising:
a test tube element comprising a vacuum facilitating an extraction of blood from a patient, and further comprising a test tube septum; and
a heat transfer element encapsulating the test tube element and storing at least two reagents capable of initiating a chemical reaction contemporaneously with the extraction of the blood from the patient, the chemical reaction exchanging heat with the blood, the heat transfer element further comprising a fracturable element that when fractured enables the at least two reagents to initiate the chemical reaction; and wherein a perimeter of the heat transfer element includes insulation inhibiting a loss of a temperature change of the blood resulting from the heat exchange resulting from the chemical reaction.

9. The blood collection apparatus of claim 8, wherein the chemical reaction is an endothermic reaction, and wherein the test tube septum comprises an indicator of an endothermic capability of the blood collection apparatus.

10. The blood collection apparatus of claim 8, wherein the insulation element does not interfere with the test tube element being removed from the heat transfer element.

11. The blood collection apparatus of claim 8, wherein the heat transfer element encapsulating the test tube element further comprises a reagents mixing element.

12. The blood collection apparatus of claim 8, wherein the heat transfer element further comprises an ampoule storing at least one of the at least two reagents.

13. A blood collection apparatus comprising:
a test tube element comprising a vacuum facilitating an extraction of blood from a patient, and further comprising a test tube septum; and
a heat transfer element encapsulating the test tube element and capable of initiating a heat transfer process contemporaneously with the extraction of the blood from the patient.

14. The blood collection apparatus of claim 13, wherein the heat transfer element encapsulating the test tube element stores a heat transfer agent for initiating the heat transfer process contemporaneously with the extraction of the blood from the patient.

15. The blood collection apparatus of claim 13, wherein a perimeter of the heat transfer element includes insulation inhibiting a loss of a temperature change of the blood resulting from the heat transfer process.

16. The blood collection apparatus of claim 13, further comprising an insulation element encapsulating the heat transfer element, the insulation element inhibiting a loss of a temperature change of the blood resulting from the heat transfer process; and wherein the heat transfer element encapsulating the test tube element comprises a heat transfer agent for initiating the heat transfer process contemporaneously with the extraction of the blood from the patient.

17. The blood collection apparatus of claim 13, wherein the blood collection apparatus further comprises a circumference suitable to be mated with a general purpose device selected from the group consisting of a general purpose test tube needle holder and a general purpose syringe.

18. The blood collection apparatus of claim 13, wherein the heat transfer element encapsulating the test tube element stores a heat transfer agent for initiating the heat transfer process contemporaneously with the extraction of the blood from the patient; and wherein the test tube element is a general purpose test tube removable from the heat transfer element contemporaneously with an initiation of a blood test.

19. The blood collection apparatus of claim 13, wherein the heat transfer element encapsulating the test tube element stores at least two reagents capable of initiating an endothermic reaction contemporaneously with the extraction of the blood from the patient, the endothermic reaction absorbing heat from the blood; wherein the heat transfer element further comprises an ampoule storing at east one of the at least two reagents; wherein the test tube element is a general purpose test tube removable from the heat transfer element contemporaneously with an initiation of a blood test; and wherein the blood collection apparatus further comprises a circumference suitable to be mated with a general purpose device selected from the group consisting of a general purpose test tube needle holder and a general purpose syringe.

20. The blood collection apparatus of claim 13, wherein the heat transfer element encapsulating the test tube element stores at least two reagents capable of initiating an exothermic reaction contemporaneously with the extraction of the blood from the patient, the exothermic reaction releasing heat to the blood.
